(19) United States
(12) Patent Application Publication
(10) Pub. No.: US 2013/0052750 A1
(43) Pub. Date: Feb. 28, 2013

(54) DIAGNOSTIC SYSTEM

(71) Applicant: Deborah Jean Gorth, Philadelphia, PA (US)

(72) Inventor: Deborah Jean Gorth, Philadelphia, PA (US)

(21) Appl. No.: 13/558,315

(22) Filed: Jul. 25, 2012

Related U.S. Application Data
(60) Provisional application No. 61/513,359, filed on Jul. 29, 2011.

(51) Int. Cl. G01N 33/53

(52) U.S. Cl. 436/501

(57) ABSTRACT

The present invention provides, among other things, methods of creating an external marker for diagnosis and/or analysis of one or more diseases, disorders, or conditions, which may or may not otherwise have an external marker.

### CROSS-REFERENCE TO RELATED APPLICATIONS

This application claims the benefit under 35 U.S.C. §119(e) of U.S. Provisional Application No. 61/513,359, filed Jul. 29, 2011, the contents of which are incorporated herein by reference in their entirety.

### BACKGROUND

Prompt and effective diagnosis and analysis of diseases, disorders, and conditions is critical to the medical industry and to public health.

### BRIEF DESCRIPTION OF THE DRAWING

The Figure of the Drawing is for illustration purposes only, not for limitation.

FIG. 1 shows the effect of binding affinity on urine concentration. The ‘o’ shows the effect of a 16-fold increase in serum binding affinity compared to the ‘x’.

### DEFINITIONS

Administration: The term “administration” as used herein refers to delivery of an agent to a subject. Those of ordinary skill in the art, reading the present specification, will appreciate that such administration can be by any appropriate route including, for example, oral, mucosal (e.g., nasal, sublingual, vaginal, rectal, etc), topical, transdermal, parenteral, etc. In some embodiments, administration is non-invasive (e.g., is not parenteral). In many embodiments, administration is oral.

Approximately: As used herein, the term “approximately” or “about,” as applied to one or more values of interest, refers to a value that is similar to a stated reference value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Correlates with: As used herein, an entity or event “correlates with” a disease, disorder, or condition if incidence of the entity or event shows a statistically significant correlation with the disease, disorder, or condition (or a feature of the disease, disorder or condition such as incidence, prevalence, intensity, responsiveness to therapy, etc).

Disease-associated marker: As used herein, the term “disease-associated marker” refers to an entity whose presence and/or level in serum correlates with susceptibility to, presence of, and/or degree of the disease, disorder or condition.

Indicator Agent: An “indicator agent” as that term is used herein is an entity that binds to a disease-associated marker present in serum of patients suffering from or susceptible to a disease, disorder, or condition of interest. An indicator agent, furthermore, is one that naturally, after having been administered to a subject, can be obtained from (or metabolized into an agent that can be obtained from) a subject sample material, which subject sample material can be obtained non-invasively (e.g., cell scrapings, or fluid samples such as urine, tears, saliva, breast milk, mucosal secretions, etc). According to the present invention, presence and/or level of an indicator agent in the subject's sample material after administration of the indicator agent to a subject correlates with susceptibility to, presence of, and/or degree of the disease, disorder or condition in the subject, for example by revealing presence and/or level of a disease-associated marker in the subject's serum.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, or condition has been diagnosed with and/or exhibits one or more symptoms of the disease, disorder, or condition.

Susceptible to: An individual who is “susceptible to” a disease, disorder, or condition is at risk for developing the disease, disorder, or condition. In some embodiments, an individual who is susceptible to a disease, disorder, or condition does not display any symptoms of the disease, disorder, or condition. In some embodiments, an individual who is susceptible to a disease, disorder, or condition has not been diagnosed with the disease, disorder, and/or condition. In some embodiments, an individual who is susceptible to a disease, disorder, or condition is an individual who has been exposed to conditions associated with development of the disease, disorder, or condition. In some embodiments, an individual is considered to be at risk of a particular disease, disorder, or condition if that person has or has been exposed to one or more risk factors correlated across a population with a relatively higher incidence of the disease, disorder, or condition.

### DETAILED DESCRIPTION OF CERTAIN PARTICULAR EMBODIMENTS

The present invention provides diagnostic systems that permit non-invasive diagnosis of medical conditions, for example by analysis of a bodily fluid (e.g., urine) that is secreted by an organism. Provided systems permit diagnosis through analysis of a secreted bodily fluid when the relevant condition does not itself produce or alter level or activity of an indicator marker in the secreted bodily fluid.

Among other things, the present invention provides systems including indicator agents (IA) and standardization agents (SA). In many embodiments, these agents are characterized by availability after administration via a non-invasive route; in many embodiments, one or both such agents is characterized by oral availability. Moreover, the IA is characterized in that it binds specifically to a disease-associated marker in serum of patients with a condition of interest. The SA is characterized in that it is a predominately inert molecule useful to standardize the urinated IA concentration to account for differences in gut absorption and renal clearance. In accordance with the present invention, due to its interaction with the disease-associated marker, the IA is cleared at a different rate in patients with the disease than those without. By contrast, clearance of the SA is not affected by disease state. Thus, according to the present invention, comparison of clearance rates of IA and SA can establish presence or extent of the relevant disease, disorder or condition. The present invention encompasses the recognition that a central difficulty of diagnosing medical diseases, disorders, or conditions is the lack of disease-associate markers that can act as external indicators of the condition. The renal system prevents the excretion of many proteins that could indicate a disease state. Cheap pregnancy tests work because there is a urinated marker (human chorionic gonadotropin) that indicates pregnancy, a paper-based assay can therefore be used to diagnose pregnancy by measuring the concentration of hCG in the urine. The present invention encompasses the recognition that it would be desirable to develop an analogous type of non-invasive test, for example detecting concentration of a urine component, to diagnose other diseases, disorders, and conditions. The present invention provides systems that permit such testing even for diseases, disorders and conditions that do not have urinated indicators. The present invention provides the insight that secreted (into urine or other sources that can be obtained non-invasively) indicators correlated with disease state can be developed for any disease, disorder, or condition through use of an IA whose secretion/clearance is modified based on presence or level of a disease-associated marker (that is not secreted), and in particular through comparison of IA clearance with that of an SA whose secretion/clearance is not altered by disease state.

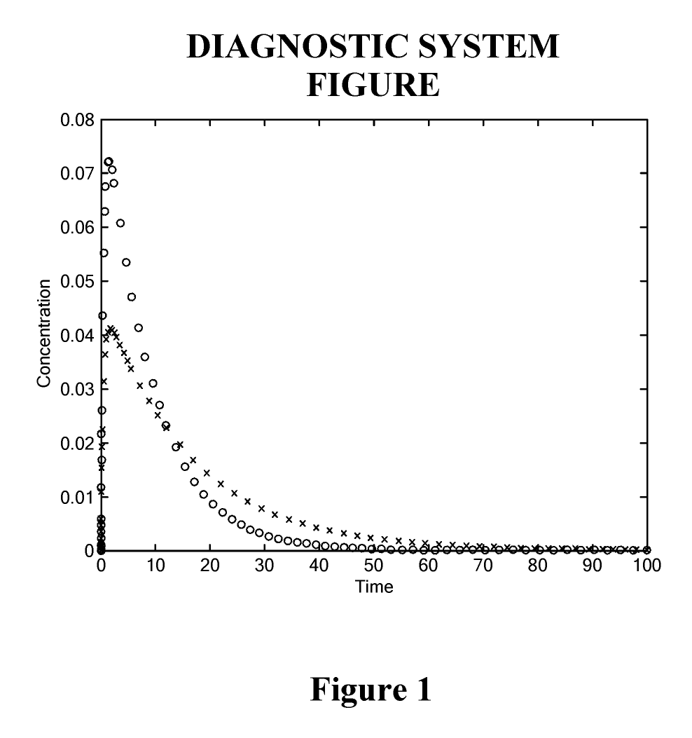

FIG. 1 below shows a particular example of different residence times of a molecule due to an increased binding affinity for an internal molecule. Where the internal molecule is a disease-associated marker, a molecule whose residence time is altered in the presence of the internal molecule is potentially useful as an IA as described herein. Although eventually all of the IA may be cleared, there is window of time that the secreted (e.g., urine) concentration of IA can be used to indicate a disease state (1-10 in FIG. 1).

In accordance with the present invention, use of an SA together with the IA can account for the user-to-user variability of gut absorption, hydration state and renal clearance, among other things. Users with a slower gut absorption rate will absorb both the IA and SA at the same retarded rate allowing the final IA/SA ratio to still indicate disease state instead of a difference in gut absorption. An increased plasma volume will dilute the concentration of the IA in the urine, but the SA will also be diluted allowing the IA/SA ratio to indicate disease state.

In some embodiments, appropriate IA can be identified using standard technologies. For example, use of high throughput binding assay screening will identify candidate agents that have an increased binding affinity for serum of patients with a given condition. The library of agents screened can be chosen from already FDA approved compounds to speed the approval process. However those skilled in the art will readily appreciate that any collection of chemical entities could be tested to identify appropriate IA.

According to the present invention, agents useful as IA are those that are cleared after administration (e.g., after administration by a non-invasive route, and in some particular embodiments after oral administration) so that they are detectable in a fluid or other source that is secreted or otherwise obtainable by non-invasive means. Preferably, the IA is cleared into urine. Moreover, agents useful as IA in accordance with the present invention are those whose extent of such clearance correlates with presence or extent of a disease, disorder, or condition as described herein.

Appropriate agents for use as SA in accordance with the present invention can similarly be determined according to known techniques. In general, useful potential SA are predominately inactive when administered (e.g., non-invasively, and particularly orally) to an organism, e.g., a human. In some embodiments, an SA is of a similar chemical class as the IA; in other embodiments, the IA and SA are structurally unrelated.

According to the present invention, agents useful as SA are those that are cleared after administration (e.g., after administration by a non-invasive route; in many embodiments after oral administration) so that they are detectable in a fluid or other source that is secreted or otherwise obtainable by non-invasive means. Preferably, the SA is cleared into the same source as is the IA, for example into urine. Moreover, agents useful as SA in accordance with the present invention are those whose extent of such clearance is not materially altered by presence or extent of a disease, disorder, or condition as described herein. Those skilled in the art will readily be able to assess what degree of change in clearance can be tolerated in a useful SA. In some embodiments, SA clearance does not change by more than 1%, 2%, 3%, 4%, 4%, 6%, 7%, 8%, 9%, 10%, 15%, 20%, 25%, 30%, or so based on presence or extent of a disease, disorder, or condition.

In accordance with the present invention, identified IA and SA are useful to diagnose susceptibility to, presence of, and/or extent of a disease disorder or condition by after simultaneous or sequential administration of these agents to an individual in need of diagnosis. Desirably, administration is by a non-invasive route; in many embodiments, administration is oral. Also desirably, only low levels of one or both such agents are required. The ratio of IA and SA cleared is then determined.

In some embodiments, a colorimetric assay is used to detect IA and/or SA. In such embodiments, IA/SA concentration ratio can be measured using existing technology, for example by having the concentration of IA and SA measured by two different assays generating two different pigments (e.g.,: red and blue) in the same region. Variations IA/SA ratio will change the color of the read out (red-purple-blue) and changes in concentration dependent on sample (e.g., urine) volume will change color saturation not the color of the read out.

### Scope and Equivalents

Those of ordinary skill in the art, reading the present disclosure will appreciate that any of a variety of particular chemical entities can be utilized as IA and/or SA. Those of ordinary skill in the art, reading the present disclosure will further appreciate that any of a variety of detection/assay systems can be utilized to determine IA and/or SA levels and/or IA/SA ratio, in subject samples.

Those of ordinary skill in the art, reading the present disclosure, will also appreciate that IA and/or SA can be detected in any of a variety of bodily samples, desirably samples that can be obtained non-invasively (e.g., cell scrapings, or fluid samples such as tears, saliva, urine, milk, mucosal secretions, etc), but that urine is of particular interest and utility.

Those of ordinary skill in the art, reading the present disclosure will still further appreciate that the teachings described herein are appropriately applicable to any disease, disorder or condition, and can be used to assess any of a variety of features of such a disease, disorder or condition including, for example, susceptibility to, presence of, or extent of the disease, disorder, or condition. Extent of a disease, disorder or condition can be assessed, of course, before, during or after therapy, so that in some embodiments the present invention provides systems for assessing, for example, responsiveness to therapy.

Those of ordinary skill in the art, reading the present disclosure will yet further appreciate that IA, SA, and/or other appropriate reagents for use in the practice of the present invention may be provided in any appropriate format, including as kits or as individual reagents.

The scope of the present invention is provided by the appended claims.

What is claimed is:

1. A system comprising:
an indicator agent (IA) that, upon administration to a subject, is cleared by the subject into a bodily sample obtainable without invasion of the subject's body, which indicator agent is characterized in that its extent of clearance correlates with presence or extent of a disease, disorder, or condition; and
a standardization agent (SA) that, upon administration to a subject, is cleared by the subject into a bodily sample obtainable without invasion of the subject's body, which standardization agent is characterized in that it is a predominately inert molecule such that its extent of clearance is not materially altered based on presence or extent of the disease, disorder, or condition;
the IA and SA being selected such that determination of IA/SA ratio in the bodily fluid can be used to diagnose the presence or extent of the disease, disorder, or condition.

2. A method comprising steps of:
administering to a subject both:
an indicator agent (IA) that, upon administration to a subject, is cleared by the subject into a bodily sample obtainable without invasion of the subject's body, which indicator agent is characterized in that its extent of clearance correlates with presence or extent of a disease, disorder, or condition; and
a standardization agent (SA) that, upon administration to a subject, is cleared by the subject into a bodily sample obtainable without invasion of the subject's body, which standardization agent is characterized in that it is a predominately inert molecule such that its extent of clearance is not materially altered based on presence or extent of the disease, disorder, or condition;
obtaining from the subject the bodily sample;
determining IA/SA ratio in the bodily sample, which IA/SA ratio can be used to diagnose the presence or extent of the disease, disorder, or condition.
